(19) United States
(12) Patent Application Publication
(10) Pub. No.: US 2010/0062474 A1
(43) Pub. Date: Mar. 11, 2010

(54) METHOD FOR THE PURIFICATION OF AT LEAST ONE TARGET SUBSTANCE THAT IS TO BE IDENTIFIED

(76) Inventor: Jochen Peter, Weilheim (DE)

Correspondence Address: JENKINS, WILSON, TAYLOR & HUNT, P. A., Suite 1200 UNIVERSITY TOWER, 3100 TOWER BLVD.,, DURHAM, NC, 27707, US

(21) Appl. No.: 12/447,772

(22) PCT Filed: Nov. 7, 2007
(86) PCT No.: PCT/EP07/09648

§ 371 Date: Apr. 29, 2009

(30) Foreign Application Priority Data
Nov. 8, 2006 (DE) 10 2006 052 923.5
Dec. 21, 2006 (DE) 10 2006 060 717.1

(51) Int. Cl. C12Q 1/02

(52) U.S. Cl. 435/29

(57) ABSTRACT

Disclosed is a method for purifying at least one target substance that is to be identified and is present or is formed in a cell culture medium when cells are cultivated. In said method, magnetic particles, i.e. beads, to the functionalized surface of which the target substance selectively attaches, are added to the cell culture medium, and the particles to which the target substance is attached are selected out of the cell culture medium by applying a magnetic field. The method is characterized by the following steps: a serum substitute is provided that is obtained from a natural serum and is free or virtually free of low-molecular substances having a maximum mass of 60 kDa, particularly a maximum mass of 10 kDa; the serum substitute is admixed to the cell culture medium which already contains the cells or to which the cells are added; the cells are incubated in the cell culture medium enriched with serum substitute; at least some of the cell culture supernatant formed during the incubation is separated; the cell culture supernatant is filtered by means of an ultrafiltering process so as to obtain a retentate; the beads are supplied in such a way that the functionalized surface of the beads comprises a plurality of dendrimers containing up to 10 branches, i.e. ten generations, the terminal points of the last generation of each dendrimer being modified; the beads and the retentate are admixed to a buffer solution so as to obtain a mixture; the target substances contained in the retentate are incubated and are fixed to the beads; and the magnetic beads are magnetically selected out of the mixture.

### TECHNICAL FIELD

The invention relates to a method for purifying at least one target substance to be identified which is present or is formed in a cell culture medium when cells are cultured, in which magnetic particles, which are known as beads, which are functionalised on the surface thereof and to the surface of which the target substance selectively attaches, are added to the cell culture medium, and the particles to which the target substance is attached are selected from the cell culture medium by applying a magnetic field.

### PRIOR ART

Generic methods are preferably used for the preparative isolation of substances which are released by cells in a cell culture medium in order to study cell properties and the further growth behaviour thereof. A particularly relevant application is the investigation of tumor cells, the uncontrolled cell growth of which is the commonest cause of death in humans.

In order to provide a responsible treatment for fighting a tumor detected in humans, it is necessary to initially determine the type of tumor, for example whether it is a non-invasive or invasive type of tumor or is a ductal carcinoma in situ, etc. Subsequent treatment depends on the type of tumor since tumor cell progression causes the properties of tumor cells to change. For example, the non-invasive cancer cell line MCF-7 contains an oestrogen receptor, so this type of tumor can be treated by an oestrogen receptor inhibitor, for example tamoxifen. During progression, a benign, i.e. non-invasive, tumor cell can transform into a malignant, i.e. invasive, type of cell, which no longer has the oestrogen receptor on its surface, thus precluding the possibility of hormone treatment.

Tumor progression is basically triggered by mutations which may in turn be caused by external effects such as a lack of oxygen (hypoxia). In addition to the fact that tumor cell surfaces have, inter alia, receptors which experience major changes in type, effect and quantity due to mutation, tumor cells also change their behaviour in relation to the secretion of different regulatory peptides and proteins.

One approach for identifying tumor markers from serum/plasma in modern proteome research is to use various chromatographic steps, i.e. two-dimensional gel electrophoresis and, subsequent mass spectrometric methods. Currently, however, the search for potential markers has stalled on account of the markers being masked by very highly concentrated proteins in the serum/plasma. For this reason, tests have been carried out more and more on tumor cell lines. These tests on tumor cells have shown, however, that peptides and proteins are released by cells only at extremely low concentrations, i.e. amounts of 10⁻¹² to 10⁻⁹ M. Since the detection limit of current mass spectrometers is 10⁻⁹ M and the substances must be present in a highly pure state in order to be detected by a mass spectrometer, efficient purification steps are necessary to concentrate analytes in cell culture supernatants, for example secreted molecules.

For this reason, attention in the field of proteome research is currently focussed on the pre-analysis of substances present at low concentrations. A series of different successive purification steps carried out on a low amount of protein leads, however, to a severe to total loss of protein on account of the fact that it forms unspecific bonds with the walls of the vessel to be used. An additional problem encountered when performing a large number of purification steps is that the samples become increasingly contaminated and thus make mass spectrometric analysis impossible on account of interfering signals. For this reason, cell secretion tests have only been able to be carried out with very high cell counts.

It has been found that experiments with an initial cell count in the order of magnitude of 10⁷ cells, and a multiplication phase lasting a plurality of days in a serum-containing medium, followed by incubation in a serum-free environment produce only a few mg of total proteins. For example, tests on yeast cells after completion of the cell culturing process with a cell count of 10⁹ cells measured a concentration of 30 nM AIP proteins in the supernatants. However, such large amounts of cells are generally not available for analysis. For example, in tests on cancer cells which have been removed from tumor tissue by biopsy, a maximum of some 10⁵ cells can be isolated per needle core sample, but many cells contained therein are not tumor cells. Due to the very low concentrations of the distributed substances it is therefore extremely important that the secretion molecules be isolated efficiently in a single purification step and the subsequent analysis procedure be carried out without any intermediate steps.

In the last five years, attempts have been made to use SELDI-TOF MS technology (surface enhanced laser desorption ionisation time of flight mass spectrometry) as a method for screening the peptides and proteins secreted by cells. In this approach, the samples to be tested are placed on a 3 mm² chip with a mostly hydrophobic surface. The proteins bound on the chip are subsequently ionised by laser bombardment, detected in a mass spectrometer and visualized subsequently in on a two-dimensional graph. This method enables different cancer cell lines to be compared with one another for example. A limiting factor is, however, the insufficient sensitivity of this technology. The sensitivity limit for this system is generally 300 nM at most and so, at present, the SELDI-TOF MS technology is still only used for profiling serum samples.

Sensitivity can only be increased by using particles of smaller sizes since this provides a greater total surface area to bind proteins. Using magnetic particles as carriers for the binding of proteins and peptides enables this process to be automated and thus achieve a greater sample throughput. The use of magnetic particles, magnetic beads as they are known, to isolate proteins has been demonstrated with biotinylated antibodies attached to magnetic streptavidin beads, and has been used successfully in mass spectrometric tests on the prostrate specific antigen (PSA) obtained from serum. However, when using magnetic “antibody particles” only one protein can be isolated at a time.

Particles with hydrophobic surfaces have been found to be expedient for binding as many different peptides or proteins as possible from a solution such as plasma, serum, urine or cell culture medium. These particles bind proteins by hydrophobic interaction and can therefore bind a wide range of peptides or proteins. This type of magnetic microparticles with hydrophobic surfaces (RP-Beads) is thus being used increasingly for desalination and for concentrating peptides and proteins from serum for example. These purified peptides and proteins obtained in this way from different serums for example are now typically applied directly to a MALDI-TOF MS target plate in order to produce peptide and protein profiles for example.

Serum is composed of a large number of substances which are present in greatly varying concentrations. For example, albumin is present in concentrations in the mg/ml range, whereas interleukin is only present in concentrations in the ng/ml range. These highly concentrated peptides and proteins prevent the ability to detect peptides and proteins at low concentrations, like those released into the cell culture medium from cells, for example tumor cells, growing in a cell culture. Regulatory peptides and proteins of this type are released only in very small amounts.

Moreover, cells also generally require fetal calf serum (FCS) to be added in order to grow in a cell culture medium. On the one hand, this additive provides the cells with the required growth factors, but on the other hand this means that further additional proteins at very high concentrations are added. During the process of bonding to hydrophobic magnetic beads, these highly concentrated substances therefore compete for the binding sites, and so, peptides and proteins at low concentrations are only bound to a small fraction of these binding sites.

The magnetic beads which have been used successfully to analyse serum or plasma samples are therefore only suitable to a limited extent for use in a cell culture medium for selectively separating regulatory peptides and proteins which have been released by tumor cells into the cell culture medium.

### DISCLOSURE OF THE INVENTION

The object of the invention is to develop a method for purifying at least one target substance which is to be identified and is released by cells in a cell culture medium, in which method magnetic particles, beads as they are known, with different functionalised surfaces, are added to the cell culture medium of which the target substance selectively attaches to the bead surface, are selected from the cell culture medium by applying a magnetic field, in such a way that it should be possible to selectively separate the target substance out of the cell culture medium with an improved degree of selectivity, the target substance being present in the cell culture medium in a low amount or concentration. In particular, the purification method should extract, in a targeted manner, substances present at low concentrations between 10⁻¹² and 10⁻⁷ M in the cell culture medium as a result, inter alia, of secretory processes, when cells are incubated, in particular tumor cells.

The object of the invention is achieved by the method specified in claim 1. The method according to the solution is also advantageously developed by the features specified in the sub-claims and those which can be inferred from the description, in particular with reference to the embodiments.

The method according to the invention for purifying at least one target substance to be identified, preferably regulatory peptides and proteins, which are released by cells, in particular tumor cells, in a cell culture medium, involves (a) carrying out the cell culture using a serum substitute and (b) adding to the cell culture medium magnetic surface-functionalised particles, magnetic beads as they are known, to the surface of which the target substance selectively attaches, and selecting particles to which the target substance is attached out of the cell culture medium by applying a magnetic field, and is characterised by the following method steps: (The key words for each individual main process steps are provided in bold to make the steps easier to follow).

Providing a serum substitute which is obtained from a natural serum and is free or virtually free of low-molecular substances having a molecular mass less than or equal to 60 kDa, 30 kDa, preferably less than or equal to 10 kDa. The serum substitute is thus characterised specifically by the fact that, like other products, it does not have an adverse effect on cell growth but in particular does not contain any disruptive substances less than 10 kDa in size.

In principle, animal serums may be used as the natural serum, preferably fetal calf serum, bovine or ovine serum, to name but a few. Human serum may also be used.

In order to prepare and provide the serum substitute, in a preferred embodiment the natural serum is initially centrifuged by using ultrafiltration units to obtain a supernatant, the retentate as it is known, and a permeate.

Depending on the filter size used, the permeate contains substances with particle sizes smaller than the filter pore size used in each case. For this purpose, membranes with suitably selected pore sizes or exclusion limits between 3 kDa and 100 kDa are used. In order to obtain a serum which is as free as possible of tiny particles, a growth medium is added to the supernatant obtained from the first centrifugation process and centrifugation is carried out again. This procedure, referred to below as a washing procedure, is advantageously repeated a plurality of times. Growth media, such as Dulbecco's Modified Eagle Medium (DMEM), IMDM, IMEM, ERDF, RPMI1640 or buffers, such as PBS, MES, TRIS, etc. may be used as the wash solution which is to be added to the supernatant obtained in each case.

After the washing procedure, the supernatant or retentate obtained is subsequently back-centrifuged, in which process the vessel containing the supernatant is merely turned upside down and centrifuged again, the retentate being conveyed out of the centrifugation unit for further processing and being laced with the respective growth medium or buffer used, preferably by adding enough growth medium to obtain the original volume used. The supernatant obtained in the aforementioned manner, referred to as the serum supernatant, is subjected to a sterile filtration process for the purposes of further purification and sterilisation, preferably by using a 0.2 μm filter, and, as the serum substitute to be provided by the method according to the solution, is subsequently separated into usable amounts, 500 μl aliquots for example, and is frozen at −20° C. to −80° C. for future use.

Adding the serum substitute described above to the cell culture medium to which the cells have been added, for which process the growth media described above, i.e. Dulbecco's Modified Eagle Medium (DMEM), IMDM, IMEM, ERDF, RPMI1640 are suitable. The serum substitute is added in a volume fraction of 1 to 10%, in which the cells are incubated for a plurality of days, i.e. 1 to 14 days. A cell culture supernatant, in which substances have become concentrated during incubation of the mixture of the cell culture medium, cells and serum substitute, is subsequently separated and is transferred into appropriately formed containers, vials as they are known, and optionally frozen for the purposes of transport or storage.

For further processing or purification of the substances contained in the cell culture supernatant, the cell culture supernatant, after thawing as required, is centrifuged using an ultrafiltration unit. In this case, membranes with suitable selected pore sizes or exclusion limits of between 3 kDa and 60 kDa are preferably used. The filtrate obtained by filtration is subsequently purified by treatment with suitably functionalised magnetic beads.

For successful use of the method according to the invention, functionalised magnetic particles, beads or magnetic beads for short, in particular are to be supplied which, in comparison to beads which have been commercially available until now, have a higher binding capacity and enable a new method for binding substances to be used, which is characterised by a greater concentration of the substances at low concentrations. The functionalised surfaces of the novel magnetic beads thus have a plurality of dendrimers, each comprising up to 10 branches, i.e. 10 generations, the terminal points of the last generation of each dendrimer being modified.

Novel magnetic beads of this type can be produced or formed in the following way:

Firstly, magnetic particles, which can be obtained commercially in different particle sizes on the nano- or micro-scales, i.e. one or more nm or μm, and on the surface of which amino groups for example are applied, are to be provided. The beads are added to an organic solvent to which methyl acrylate has been added to further functionalise the amino bead surface. After an exposure time of preferably between 2 and 48 hours, the beads are separated from the reaction liquid and are washed with fresh solvent. The beads prepared in this way are subsequently added to an organic reaction liquid to which ethylenediamine has been added, to form a first dendrimer generation (G1-beads) on the surface thereof. The beads react for a further 2 to 48 hours in this environment, the reaction process being boosted by agitating the reaction bath. The beads are subsequently separated from the reaction liquid, preferably using a magnet, and washed again. In order to obtain multiple dendrimer formation, which brings about the extremely high binding capacity of the beads functionalised in the novel manner, the process described above for forming the first dendrimer generation G1 is to be repeated up to nine times.

Any dendrimer beads obtained after appropriate processing are suspended in a solvent and activated alkyl (C₁-C₃₀) or aromatic derivatives, monoaromatics or polyaromatics are added in order to finally modify the dendrimer chain terminal points with hydrophobic derivatives, thus providing the beads with hydrophobic properties. These functionalised beads are therefore termed dendritic reversed-phase beads (G1 RP beads to G10 RP beads).

The beads are supplied by separating them from the reaction suspension, washing them again and storing them in distilled water or buffer, such as PBS, MES, TRIS, etc.

For the purposes of further purification and separation of the substances contained in the sterile filtered cell culture supernatant, including the regulatory peptides and proteins secreted by the cells, the dendrimer RP beads obtained in above manner are added to the filtrate obtained after filtration and a buffer solution is also added. An incubation process is subsequently carried out at ambient temperature with a residence time from 10 to 120 minutes in duration. In this process, the substances contained in the cell culture supernatant are bound to the modified terminal points of the dendrimer structures. The magnetic beads are subsequently magnetically selected from the mixture and the substances which are bound to the dendritic structure, in particular the substances secreted by the cells, specifically the regulatory peptides and proteins, are eluted. It is of course possible to also select further substances in a targeted manner, for example metabolites, steroids, etc. depending on the functionalisation of the magnetic beads.

The core idea behind the purification method according to the invention basically involves two advantageously cooperating aspects, i.e. on the one hand the provision of a cell culture additive or serum substitute which, at the outset, does not contain any interfering substances of a small particle size which may potentially compete with the actual target substances to be identified in the subsequent analysis procedure, and on the other hand novel magnetic beads with a dendritic structure and a reversed phase, whereas the dendritic structure having up to ten branch points and thus optimising the number thereof for binding target substances to be identified.

The method according to the solution enables far more substances to be isolated than the normal serum conventionally used until now, such as fetal calf serum. The use of the novel beads enables sensitivity with respect to cell culture supernatants for example to be increased by 10 to 100 times in comparison to commercially available beads. In summary, the use of the beads and the serum substitute enables sensitivity up to 1,000 times greater to be achieved than the use of conventional serum and commercially obtainable beads.

Some concrete embodiments of the process are described below:

### EXAMPLE 1

### Preparation of a Serum Substitute

(a) Low-molecular substances are removed e.g. from fetal calf serum or human serum by centrifuging and washing it with Dulbecco's Eagle Modified Medium. The centrifugation process is carried out using a filtration membrane with a pore size of between 3 kDa and 100 kDa, depending on requirements. The centrifugation process is carried out for one hour. If required, protease inhibitors (PMSF, EDTA, protease inhibitors in the form of individual inhibitors or a protease cocktail thereof, etc.) can be added to the serum before processing.
(b) Dulbecco's Eagle Modified Medium (DMEM) is added to the supernatant obtained by means of centrifugation and centrifugation is again carried out for one hour (serum purification).
(c) The centrifugation and washing step is carried out between 2 and 5 times in total.
(d) The supernatant obtained is then back-centrifuged into a vessel and is returned to the original volume used by adding DMEM.
(e) The serum replacement (also referred to as serum supernatant) obtained is sterile filtered by using a 0.2 μm filter.
(f) The serum substitute is divided into 500 μl aliquots and frozen at −20° C. until use.

### EXAMPLE 2

### Production of Modified Magnetic Beads

Starting from the amino surface of the beads, a dendritic structure is formed by carrying out a branched-chain reaction on each amino group. This process is based on the principle of synthesis of starburst molecules and is carried out until 1 to 10 branches are formed in the dendritic structure so to obtain what are known as G1 to G10 beads. At the end of the process, the terminal NH₂ groups of the dendrimer beads are modified by alkyl chains of different lengths (C₁-C₃₀) via an activated group by means of, inter alia, microwave synthesis.
(a) Provide 10 to 2,000 mg of amino beads and wash them in 10 to 100 ml MeOH in a container.
(b) Add beads to 10 to 100 ml MeOH and add 10 to 100 ml cold methyl acrylate. Allow to react at ambient temperature in an agitator for 2 to 48 hours.
(c) Separate the beads from the reaction liquid with a magnet and wash the beads 3 times with 10 to 200 ml MeOH each time.
(d) Add 10 to 100 ml MeOH and gradually add 10 to 100 ml cold ethylenediamine. Allow to react at room temperature for 2 to 48 hours while being agitated; (G1 beads).
(e) See step (c), i.e. separate the beads from the reaction liquid with a magnet and wash beads 3 times with 10 to 100 ml MeOH each time.
(f) Repeat steps (b) to (e) n times, n being 0 to 9.
(g) Wash the dendrimer beads 3 times with 10 to 100 ml MeOH each time and 5 times with 10 to 100 ml halogenated hydrocarbons each time.
(h) Suspend dendrimer beads in 50 to 100 ml halogenated solvent and gradually add 5 to 10 ml of an activated alkyl derivative, for example C₁-C₃₀ or aromatic derivatives, dropwise. React for 10 to 120 minutes at ambient temperature in an ultrasonic bath and/or by heating to a maximum of 38° C. or by means of microwave synthesis.
(i) Wash modified dendrimer beads 5 times with 10 to 100 ml MeOH each time and 5 times with 10 to 100 ml of ultrapure water.
(j) Add the dendrimer beads to ultrapure water and store at +4° C.

Alternatively, in place of method step (h), it is also possible to modify the dendrimer beads differently at the terminal points of the dendrimer chains thereof, for example by adding antibodies. For this purpose, the pre-processed dendritic amino beads (G1-G10) are reacted in a buffer with a bifunctional linker for amino groups. Glutaric dialdehyde, bis(sulphosuccinimidyl) suberate, succinimidyl suberate, dimethyl pimelimidate or bifunctional PEG linkers are preferably suitable as linkers. The beads are subsequently washed, preferably in a phosphate buffer. The antibodies are finally added to the buffer at a concentration of 1 mg/ml to 50 mg/ml of buffer. After an incubation period of approximately 1 to 14 hours at ambient temperature or +4° C., the beads provided with antibodies are washed and stored at approximately +4° C. in a buffer for subsequent use.

### EXAMPLE 3

### Purification of Substances Secreted by Tumor Cells Present in a Cell Culture Medium by Using the Serum Substitute Obtained in Example 1 and the Magnetic Particles Modified in Example 2

The tumor cells are added, depending on the scale of the experiment, to Dulbecco's Modified Eagle Medium (DMEM) and 1 to 10% serum substitute obtained from foetal calf serum (FCS) in different containers and incubated for a plurality of days at 37° C. and 5% CO₂ enrichment. The cell culture supernatant, i.e. DMEM+1 to 10% FCS serum substitute, CCS for short, is decanted into pre-treated vials and frozen at −20° C. for transport purposes.

Before purification is carried out using the modified dendrimer beads (G1-G10), the thawed CCS is filtered through a membrane filtration unit. The filtrate is subsequently purified using the modified dendrimer beads (G1-G10) and concentrated. This is followed by elution and further processing. In particular:
(a) Add cells to DMEM and FCS supernatant and, depending on the batch (volume), incubate in suitable cell culture vessels at 37° C. and 5% CO₂ for 3 days.
(b) Discard the cell culture supernatant and carefully wash the cells with 10 ml PBS at pH 7.2 or DMEM each time; repeat the washing procedure 2 to 5 times.
(c) Add the required amount of DMEM+1-10% FCS serum supernatant and incubate the cells for the required amount of time (1 to 14 days). After incubation is complete, transfer CCS into pre-treated vials (1 ml) and freeze at −20° C. or −80° C.
(d) Thaw the frozen CCS and transfer each 500 μl amount into a filtration unit.
(e) Centrifuge the CCS for 1 hour at 12,000 rpm and collect filtrate.
(f) In the meantime, prepare beads for incubation. Add 80 μl of the modified dendrimer beads (G1-G10) to 450 μl buffer at pH 7.2 and suspend for a short period of time. Subsequently separate the beads using a magnet.
(g) Add 450 μl buffer at pH 7.2 and suspend beads. Subsequently add 450 μl of the filtrate obtained by centrifuging the CCS and apply by pipette and pipette off 10 times (completely suspend beads).
(h) Incubate for 10 to 120 minutes at ambient temperature.
(i) Discard solution after magnetic separation and wash magnetic dendrimer beads (G1-G10) 3 times with 500 μl buffer each time.
(j) Add 20 μl acetonitrile:TFA 1:1, v:v and mix the bead solutions by pipetting and pipetting off 10 times (elution).
(k) Incubate for 1 to 120 minutes at ambient temperature.
(l) Separate the magnetic particles and separate afterwards elution solution for further processing.

### EXAMPLE 4

### Five Alternative Examples for Further Tests on the Magnetic Beads Provided with the Target Substances, as Obtained in the Purification Method According to the Solution

### First Alternative:

The magnetic beads loaded with the target substances are added to a non-reducing or reducing gel electrophoresis sample buffer consisting of TRIS, urea, thiourea, LDS, SDS or CHAPS, etc. The mixture thus formed is preferably heated to 60° C. for 5 to 30 minutes, causing the target substances to be released from the beads. The magnetic beads are subsequently separated from the mixture by a magnet and the remaining gel electrophoresis sample buffer with the target substances is pipetted off and added to gel slots in an electrophoresis gel (SDS gel, native gel, 2-D gel) and an electrophoretic separation process is subsequently carried out.

### Second Alternative (Only Applies to Antibodies Coupled to Dendrimer Beads (G1-G10)):

The target substances bound to the antibody particles are eluted by adding a carboxylic acid or a chaotropic high salt buffer.

The carboxylic acid eluate obtained from the elution process may be reduced in volume (speed-vac), and redissolved in an appropriate buffer solution.

Finally, the target substances contained in the buffer solution are enzymatically digested. The digested target substances are subsequently subjected to a mass spectrometric identification process by peptide mass fingerprinting (PMF) or MS/MS spectra.

### Third Alternative:

The magnetic beads loaded with one or more target substances are washed and subsequently added to a buffer solution containing a protease for digesting the target substances. The digested target substances are subsequently subjected to a mass spectrometric identification process by peptide mass fingerprinting (PMF) or MS/MS spectra. In addition, the magnetic beads are washed again and then added to a mixture of an organic solvent and/or carboxylic acids to obtain an eluate. Mass spectrometric measurements are finally carried out on the eluate.

### Fourth Alternative:

The target substances bound to the antibodies are eluted by adding a carboxylic acid or a high salt buffer. The dendritic reversed-phase beads are added to the eluates. After incubation of between 1 and 60 minutes in duration, the beads are separated by a magnet and are subsequently washed repeatedly with a buffer. It is then possible to either carry out the method described in Alternative 1 for gel electrophoretic separation or the method described in Alternative 3 for the production of proteolytic peptides which are subsequently detected and shown by MS or MS/MS spectra.

### Fifth Alternative:

Cells, such as tumor cells, are added, depending on the experiment, to different containers and incubated for a plurality of days at 37° C. and 5% CO₂ content. The cells are subsequently washed with cell culture medium or a buffer. The cell culture medium without serum is subsequently added and incubation is again carried out for between 0 and 72 hours. The respective cell culture supernatants are transferred into vials and optionally frozen at −20° C. The substances are bound to the modified dendrimer beads by adding said beads to the thawed cell culture supernatants. The beads are subsequently washed and are subjected to gel electrophoresis, as discussed in Alternative 1, or are digested by a protease, as discussed in Alternative 3, and the digested substances are subjected to an MS or MS/MS analysis.

The functionalised magnetic beads described above represent, as such, a product which can be used in principle as a substance which can affect detection in a large number of different applications. One possible use is the purification of target substances described above. In order to produce a product of this type in a general form the following measures should be taken:
a) providing magnetic particles, beads for short, with a particle size on the nano- and/or micro-scale, i.e. one or more nm or μm, and comprising functional groups such as amino groups which are applied to the surfaces thereof,
b) reacting the beads in an organic solvent containing a branching reagent such as methyl acrylate,
c) separating the beads from the organic solvent and washing the beads with an organic solvent,
d) reacting the beads with an organic liquid containing a binder such as ethylenediamine,
e) separating the beads from the organic liquid and washing the beads,
f) repeating steps b) to e) n times,
g) suspending the beads in a solution containing at least one derivative, and
h) separating and washing the beads and supplying the derivatised beads in water or buffer-containing solution.

n is preferably selected from 0 to 9. Alcohol is particularly suitable as a reaction liquid. To enable the beads to assume reversed-phase properties, said beads are derivatised by a chemical group such as alkyl (C₁-C₃₀) or aromatic groups, or proteinogenic groups. Stable magnetic particles with a particle size of between 10 nm and 10 μm are suitable as the base elements for the beads.

The group applied to the magnetic particles for the purposes of functionalising said beads is preferably composed of at least one substance of the following groups: primary and secondary amino, primary, secondary and asymmetric hydrazine, azide, phosphine, aldehyde, polyaldehyde, carboxylic acid, carboxylic acid ester, cyanate, isocyanate, thiocyanate, hydroxyl, thiol, imino, hydrazide, piperidine, azomethine, semicarbazone, hydrazone, cyanobromide, tosyl, epoxide, cyanuric acid chloride and cyanuric acid ester groups and diamino, triamino and tetraamino heterocycles.

The functionalised magnetic particles described above are further reacted with branching reagents which are provided in a solvent, the branching reagents being selected from at least one of the following groups:

melamine, diamino, triamino and tetraamino heterocycles, triepoxides, tetraepoxides, diallylamines, methyl acrylate, triacrylate, tetraacrylate, tris(hydroxymethylamine), oxazines, oxetanes and diethanolamines.

The magnetic particles loaded with the branching reagent are subsequently reacted with a bi- or trifunctional linker, the linker being selected from one of the following listed substances: lysine, diaminoalkanes, diamino-, triamino-, tetraamino heterocycles, diaminoethylene glycols, piperazine, allylamines, triepoxides, tetraepoxides, tris(hydroxymethylamine).

Finally, after the aforementioned method steps b) to e) are repeated n times, magnetic particles are derivatised with the following chemical groups as terminal groups or with peptides, proteins, glycanes or glycoproteins of any type:

n-alkyl, sec-alkyl or tert-alkyl groups, unsubstituted and substituted aryl, unsubstituted and substituted acyl, primary, secondary and tertiary amino, primary, secondary and asymmetric hydrazine, azide, aldehyde, phosphine, polyaldehyde, carboxylic acid, carboxylic acid ester, carboxylic acid halogenide, carboxylic acid anhydride, cyanate, isocyanate, thiocyanate, hydroxyl, thiol, sulphide, sulphite, sulphate, imino, hydrazide, piperidine, azomethine, semicarbazone, hydrazone, hydroxamic acid, amidrazone, amidine, cyanobromide, tosyl, epoxide, cyanuric acid chloride, cyanuric acid ester groups, unsubstituted and substituted carbodiimides, N-hydroxysuccinimide ester, ethylene glycols, unsubstituted and substituted aryl alkanes, arylalkenes, heterocyclic compounds, mono and polycyclic compounds. In this case, the number of carbons is between 2 and 100.

What is claimed is:

1. A method for purifying at least one target substance to be identified which is present or formed in a cell culture medium when cells are cultivated, in which magnetic particles, beads as they are known, which are functionalised on the surface thereof and to the surface of which the target substance selectively attaches, are added to the cell culture medium, and the particles to which the target substance is attached are selected from the cell culture medium by applying a magnetic field, characterised by the following method steps:
providing a serum substitute which is obtained from a natural serum and is free or virtually free of low-molecular substances having a mass less than or equal to 60 kDa, in particular less than or equal to 10 kDa,
adding the serum substitute to the cell culture medium which already contains the cells or to which the cells are added,
incubating the cells in the cell culture medium enriched with serum substitute,
separating at least some of the cell culture supernatant formed during the incubation process,
filtering the cell culture supernatant by means of an ultrafiltration process so as to obtain a retentate,
supplying the beads in such a way that the functionalised surface of the beads comprises a plurality of dendrimers containing up to 10 branches each, i.e. 10 generations, the terminal points of the last generation of each dendrimer being modified,
adding the beads and a buffer solution to the retentate so as to form a mixture,
incubating and binding the target substances contained in the retentate to the beads and magnetically selecting the magnetic beads out of the mixture.

2. The method according to claim 1, characterised in that the serum substitute is obtained as the retentate after ultrafiltration of a natural blood serum from an animal or human, and in that the retentate is washed with a growth medium or buffer.

3. The method according to claim 2, characterised in that Dulbecco's Modified Eagle Medium (DMEM), IMDM, IMEM, ERDF or RPMI1640 is used as the growth medium wash solution.

4. The method according to claim 1, characterised in that the serum substitute is obtained and provided in the following manner:
a) centrifuging natural serum using an ultrafiltration unit so as to obtain a supernatant, the retentate as it known, and a permeate,
b) adding a growth medium to the retentate and subsequently carrying out a centrifugation process according to step a),
c) repeating step b) n times,
d) adding growth medium to the retentate obtained so as to obtain a serum supernatant as it is known, and
e) filtering the serum supernatant so as to obtain a permeate corresponding to the serum substitute.

5. The method according to claim 4, characterised in that sterile filtration is carried out when the serum supernatant is filtered.

6. The method according to claim 4, characterised in that at least one reversible or irreversible protease inhibitor is added to the natural serum before step a).

7. The method according to claim 6, characterised in that phenylmethylsulphonyl fluoride (PMSF) and/or ethylenediamine tetraacetic acid (EDTA) and/or proteinogenic inhibitors are added to the serum individually as protease inhibitors or are added as a protease inhibitor cocktail.

8. The method according to claim 4, characterised in that the centrifugation process in steps a) and b) is carried out for at least 1 hour at ambient temperature down to a temperature of +4° C.

9. The method according to claim 4, characterised in that step b) is repeated n times, n being 2 to 5.

10. The method according to claim 4, characterised in that the filtration process in step e) is carried out using a 0.2 μm sterile filter.

11. The method according to claim 1, characterised in that the cell culture supernatant is frozen at −20° C. to −80° C. before filtration, in that the frozen cell culture supernatant is thawed, and in that filtration is carried out by means of centrifugation using an ultrafiltration unit.

12. The method according to claim 1, characterised in that an antichaotropic buffer solution is used when adding the beads and the retentate.

13. The method according to claim 1, characterised in that after incubation and after the target substance contained in the retentate is bound to the beads and the beads have been magnetically selected, the beads provided with the target substance are washed with a low salt buffer solution, and in that the at least one target substance is subsequently eluted from the beads in a mixture containing at least one organic component and/or an acid.

14. The method according to claim 13, characterised in that alcohol or acetonitrile are used as the organic substance and carboxylic acid or mono-, di-, trihalogen carboxylic acid are used as the acid.

15. The method according to claim 1, characterised in that Dulbecco's Modified Eagle Medium (DMEM), IMDM, IMEM, ERDF or RPMI1640 are used as the wash solution.

16. The method according to claim 1, characterised in that 1 to 10% by volume of serum substitute is added to the cell culture medium when the serum substitute is added to the cell culture medium.

17. The method according to claim 1, characterised in that the following steps are carried out in order to supply the beads:
a) providing magnetic particles, beads for short, having a particle size on the nano- and/or micro-scale, i.e. 1 or more nm or μm, and comprising amino groups applied to the surfaces thereof,
b) reacting the beads in a methyl acrylate-containing organic solvent,
c) separating the beads from the organic solvent and washing the beads with an organic solvent,
d) reacting the beads in an ethylenediamine-containing organic liquid,
e) separating the beads from the organic liquid and washing the beads,
f) repeating steps b) to e) n times
g) suspending the beads in a solution containing at least one functionalising substance, and
h) separating and washing the beads and supplying the functionalised beads in water.

18. The method according to claim 17, characterised in that n is selected from 0 to 9.

19. The method according to claim 17 or claim 18, characterised in that alcohol, R—OH for short, is used as the reaction liquid, R being C₁ to C₆.

20. The method according to claim 17, characterised in that the suspension process is carried in an ultrasound bath and/or by heating to a maximum of 36° C. and/or by microwave synthesis.

21. The method according to claim 17, characterised in that halogen-containing solvents, to which activated alkyl derivatives (C₁ to C₃₀) are added, are used as the solution containing at least one functionalising substance, halogens, in particular Cl, Br, J, cyanates, isocyanates or isothiocyanates, being used as activated groups.

22. The method according to claim 17, characterised in that, after reaction step f), the beads are reacted with a bifunctional linker for amino groups, in that the beads are washed in a buffer, and in that proteins, e.g. antibodies at a concentration of c=1 μg/ml to 50 mg/ml in a buffer solution are used as the at least one functionalising substance.

23. The method according to claim 22, characterised in that glutaric dialdehyde, bisulphosuccinimidyl suberate (BS3), disuccinimidyl suberate (DSS), dimethyl pimelimidate (DMP) or bifunctional PEG linkers are used as bifunctional linkers.

24. The method according to claim 22, characterised in that a phosphate or carbonate buffer is used as the washing buffer.

25. The method according to claim 1, characterised in that the magnetic beads loaded with the at least one target substance are added to a gel electrophoresis sample buffer, in that the mixture thus formed is heated to 40 to 95° C. for 5 to 30 minutes, in that the magnetic beads are separated from the mixture by a magnet, and in that the remaining gel electrophoresis buffer is pipetted off and is added to gel slots of an electrophoresis gel, and undergoes electrophoretic separation.

26. The method according to claim 1, characterised in that the target substances bound to the beads can be eluted by adding a carboxylic acid.

27. The method according to claim 26, characterised in that the carboxylic acid eluate obtained from the elution process may be reduced in volume (speed-vac), and redissolved in an appropriate buffer solution.

28. The method according to either claim 26 or claim 27, characterised in that the target substance undergoes enzymatic digestion, and in that the digested target substances undergo a mass spectrometric identification process by using peptide mass fingerprinting (PMF) or MS/MS spectra.

29. The method according to claim 1, characterised in that the magnetic beads loaded with the target substances are washed and subsequently added to a buffer solution containing a protease for digesting the target substances.

30. The method according to claim 29, characterised in that mass spectrometric measurements are carried out after the enzymatic digestion process by peptide mass fingerprinting (PMF) or MS/MS spectra.

31. The method according to either claim 28 or claim 29, characterised in that the beads are washed and subsequently added to a mixture of an organic solvent and/or carboxylic acids so as to obtain an eluate.

32. The method according to claim 31, characterised in that mass spectrometric measurements are carried out on the eluate.

33. The method according to claim 1, characterised in that the at least one target substance bound to the beads is eluted by adding a carboxylic acid or a chaotropic buffer and the substances contained in the eluate obtained are bound using magnetic, dendritic, reversed-phase particles.

34. The method according to claim 33, characterised in that the magnetic, dendritic, reversed-phase particles loaded with the substances contained in the eluate are added to a gel electrophoresis sample buffer, in that the mixture thus formed is heated to 40 to 95° C. for 5 to 30 minutes, in that the magnetic beads are separated from the mixture by a magnet, and in that the remaining gel electrophoresis sample buffer is pipetted off and added to gel slots in an electrophoresis gel and an electrophoretic separation process is carried out.

35. The method according to claim 33, characterised in that the magnetic, dendritic, reversed-phase particles loaded with the substances contained in the eluate are washed and subsequently added to a buffer solution containing a protease for digesting the substances bound to the particles.

36. A product formed from a serum from which low-molecular substance components are removed by a purification method, characterised in that the purification method provides the following method steps:
a) centrifuging natural serum using an ultrafiltration unit so as to obtain a supernatant, the retentate as it known, and a permeate,
b) adding a growth medium to the retentate and subsequently carrying out a centrifugation process according to step a),
c) repeating step b) n times,
d) adding growth medium to the retentate obtained so as to obtain a serum supernatant as it is known, and
e) filtration of the serum supernatant so as to obtain a permeate corresponding to the serum substitute.

37. A product, comprising magnetic beads which have a functionalised surface and can be produced in the following manner:
a) providing magnetic particles, beads for short, with a particle size on the nano- and/or micro-scale, i.e. one or more nm or μm, and comprising functional groups such as amino groups which are applied to the surfaces thereof,
b) reacting the beads in an organic solvent containing a branching reagent such as methyl acrylate,
c) separating the beads from the organic solvent and washing the beads with an organic solvent,
d) reacting the beads with an organic liquid containing a binder such as ethylenediamine,
e) separating the beads from the organic liquid and washing the beads,
f) repeating steps b) to e) n times,
g) suspending the beads in a solution containing at least one derivative, and
h) separating and washing the beads and supplying the derivatised beads in water or buffer-containing solution.

38. The product according to claim 37, characterised in that n is selected from 0 to 9.

39. The product according to claim 37, characterised in that alcohol is used as a reaction liquid.

40. The product according to claim 37, characterised in that the beads are derivatised by chemical groups such as alkyl (C₁-C₃₀) or aromatic groups, or proteinogenic groups and thus assume reversed-phase properties.

41. The product according to claim 37, characterised in that stable magnetic particles with a particle size of between 10 nm and 10 μm are used as base elements.

42. The product according to claim 37, characterised in that the group applied to the magnetic particles is formed from at least one substance of the following substance groups:
primary and secondary amino, primary, secondary and asymmetric hydrazine, azide, phosphine, aldehyde, polyaldehyde, carboxylic acid, carboxylic acid ester, cyanate, isocyanate, thiocyanate, hydroxyl, thiol, imino, hydrazide, piperidine, azomethine, semicarbazone, hydrazone, cyanobromide, tosyl, epoxide, cyanuric acid chloride and cyanuric acid ester groups and diamino, triamino and tetraamino heterocycles.

43. The product according to claim 42, characterised in that the functionalised beads are reacted with branching reagents provided in a solvent, and in that the branching reagents are selected from at least one of the following groups: melamine, diamino, triamino and tetraamino heterocycles, triepoxides, tetraepoxides, diallylamines, methyl acrylate, triacrylate, tetraacrylate, tris(hydroxymethylamine), oxazines, oxetanes and diethanolamines.

44. The product according to claim 37, characterised in that the magnetic particles loaded with the branching reagent are reacted with a bi- or trifunctional linker, and in that the linker is selected from the following list of substances:
lysine, diaminoalkanes, diamino-, triamino-, tetraamino heterocycles, diaminoethylene glycols, piperazine, allylamines, triepoxides, tetraepoxides, tris(hydroxymethylamine).

45. The product according to claim 37, characterised in that, after steps b) to e) are repeated n times, the magnetic particles are derivatised with the following chemical groups as terminal groups or with peptides, proteins, glycanes or glycoproteins of any type:
n-alkyl, sec-alkyl or tert-alkyl groups, unsubstituted and substituted aryl, unsubstituted and substituted acyl, primary, secondary and tertiary amino, primary, secondary and asymmetric hydrazine, azide, aldehyde, phosphine, polyaldehyde, carboxylic acid, carboxylic acid ester, carboxylic acid halogenide, carboxylic acid anhydride, cyanate, isocyanate, thiocyanate, hydroxyl, thiol, sulphide, sulphite, sulphate, imino, hydrazide, piperidine, azomethine, semicarbazone, hydrazone, hydroxamic acid, amidrazone, amidine, cyanobromide, tosyl, epoxide, cyanuric acid chloride, cyanuric acid ester groups, unsubstituted and substituted carbodiimides, N-hydroxysuccinimide ester, ethylene glycols, unsubstituted and substituted aryl alkanes, arylalkenes, heterocyclic compounds, mono and polycyclic compounds.
